(19) United States
(12) Patent Application Publication
(10) Pub. No.: US 2006/0281915 A1
(43) Pub. Date: Dec. 14, 2006

(54) Acid cefotetan totally solvent-free and method for obtaining same

(75) Inventors: Maurizio Zenoni, Paullo (IT); Alessandro Donadelli, CasalPusterlengo (IT)

Correspondence Address: C. IRVIN MCCLELLAND;OBLON, SPIVAK, MCCLELLAND, MAIER & NEUSTADT, P.C., 1940 DUKE STREET, ALEXANDRIA, VA, 22314, US

(73) Assignee: ACS DOBFAR S.p.A., Tribiano (IT)

(21) Appl. No.: 11/410,022

(22) Filed: Apr. 25, 2006

(30) Foreign Application Priority Data
Jun. 10, 2005 (IT) MI2005A 001085

(51) Int. Cl. C07D 501/14

(52) U.S. Cl. 540/222

(57) ABSTRACT

The invention relates to a method for obtaining cefotetan acid substantially free of tautomer, by initial isolation of the crude product with a limited tautomer content, followed by purification through a chromatographic column. The invention also concerns the acid cefotetan totally solvent-free thereby obtained.

### BACKGROUND OF THE INVENTION

1. Field of the Invention

Cefotetan is an important antibiotic which has been used for some years in the therapy of infections from gram-negative bacteria resistant to the more common antibiotics, and is characterised by the formula

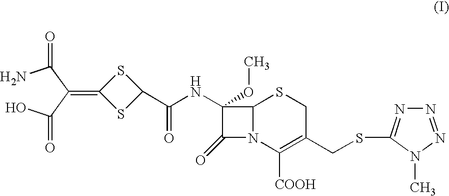

It is administered as the disodium salt by intravenous and intramuscular injection.

2. Description of the Related Art

Cefotetan is claimed in U.S. Pat. No. 4,263,432, claim 7 of which is specific for the compound of formula (I).

Claim 9 of that patent protects a tautomer thereof, which as the sodium salt is characterised by the formula

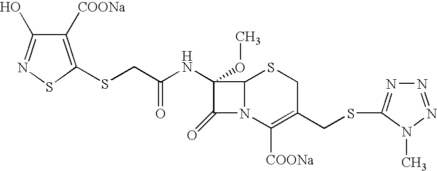

The product of formula (I) is obtained by a chemical synthesis process comprising numerous steps well known in the literature, however on termination of the process the product obtained contains a small percentage of the compound of formula (II) formed during the synthesis, its tautomerization to give the compound of formula (I) in the final solution not going to completion. Consequently, the injectable sterile solution contains the disodium salt of the compound of formula (I), together with a small quantity of the compound of formula (II). USA pharmacopea No. 28 defines a “cefotetan injection” as an isoosmotic sterile solution of the acid compound of formula (I) and of sodium bicarbonate in water for injections and one or more buffers. Analysis of cefotetan by liquid phase chromatography shows the presence of two peaks of different retention times, the solution containing not less than 90% and not more than 120% of the cefotetan declared on the label.

A study published in Chem. Pharm. Bull. 37(7) 1864-1869 (1989) shows that cefotetan tautomerization is facilitated in aqueous solution at alkaline pH. At the same time, the microbiological activity of the two tautomers in vitro appears almost equivalent, whereas their pharmacokinetic behaviour can differ, consequently the two tautomers can differ from the clinical behaviour viewpoint. The aforestated evidently demonstrates that the known art does not embrace a method suitable for nullifying or at least minimizing the content of tautomer of formula (II) in the injectable sterile cefotetan of formula (I). The importance of a method which enables cefotetan to be prepared without impurities and with a tautomer (II) content (HPLC) not greater than 0.5% is therefore evident.

### SUMMARY OF THE INVENTION

The inventors of the present application have surprisingly discovered a process by which the content of the tautomer of formula (II) can be drastically reduced by a process which does not use solvents, but only water, to hence provide solvent-free cefotetan acid free from which an injectable lyophilized product is obtained which is likewise solvent-free. This fact is particularly important considering that commercially available cefotetan acid has an overall solvent content between 1.0 and 1.5%, whereas the sodium salt obtained from it contains a total of about 0.16% of the following solvents: ethanol, methanol, methylethylketone and n-butanol.

The process of the invention enables cefotetan of formula (I) to be obtained containing up to 0.5% of the tautomer of formula (II) in its acid form with K.F up to 2.5%, dry content at least 99.0% and totally free of solvents.

The invention concerns also the acid cefotetan of formula (I) which is totally solvent-free.

This process is characterised by slowly and gradually acidifying an aqueous solution of cefotetan at pH 7.5 containing carbon dioxide and up to 5% of the said tautomer of formula (II), with an acid chosen from the group consisting of dilute hydrochloric acid and dilute phosphoric acid to pH 3.4, while maintaining the solution under agitation and under vacuum at a temperature between 20° and 35° C. in order to remove the carbon dioxide, said solution then being further acidified to pH 1.5 to allow crystallization of the cefotetan of formula (I), which is filtered off and washed, this cefotetan then being returned to solution at pH less than 7, the solution being adsorbed on HP 20 SS resin and then eluted with osmotized water to obtain a solution from which the aforesaid cefotetan of formula (I) is precipitated by acidification to pH 1.5 with dilute hydrochloric acid.

### DETAILED DESCRIPTION OF THE INVENTION

The implementation of the process will be more apparent from the ensuing detailed description of practical embodiment thereof, given by way of non-limiting example.

### EXAMPLE

An aqueous solution containing crude cefotetan is prepared by a method known in the literature, from 20 g of benzhydryl 7β-[2-bromoacetamido]-7α-methoxy-3-(1-methyltetrazol-5-yl)thiomethyl-3-cephem-4-carboxylate. The final step results in a carbon dioxide-containing solution with a tautomer content ≦5% at pH 7.5. At this point the tautomerization to cefotetan does not proceed further and then product is then usually isolated.

However, in this specific case the solution is acidified to pH 5.15 with 15% HCl at 10-15° C., controlling carbon dioxide development. The solution is maintained under agitation under a vacuum of 22 mm Hg at 20° C. and constant pH of 5.15. 1.67 g of 10% phosphoric acid is added to correct the pH to 4.5. The solution is again put under vacuum to eliminate further carbon dioxide to obtain a solution containing 12.94 g of cefotetan in 260 ml. It is decolorized with 2 g of decolorizing carbon, which is filtered off an washed with three portions of demineralized water for a total of 53 ml. The decolorized solution is at pH 5. The pH is corrected to 3.5 with 10% phosphoric acid, the temperature brought to 30-35° C. and again agitated under vacuum at 31 mmHg. The operation is repeated correcting to a constant pH 3.2 with 10% phosphoric acid and agitating under vacuum. The pH is further lowered to 2.5 with 5% HCl at 30° C. while agitating under vacuum. This procedure is continued, further lowering the pH to 1.5 with 5% HCl at 30° C. under vacuum, for 30 min.

The solution is then cooled to +5° C. and maintained under agitation for a further 30 min. The cefotetan crystallizes, is filtered off, washed with 1% HCl and then with demineralized water, both precooled to +5° C. 33 g of crude moist cefotetan are obtained with a tautomer (II) content (HPLC) of 2-10%.

The product is fed into 152 ml osmotized water at +5° C., and 4.46 g of sodium bicarbonate are added in portions while maintaining the pH ≦7. The solution obtained is brought to pH 5.5 with 5% HCl, maintaining it under vacuum to remove the residual carbon dioxide.

The solution contains 12.9 g of cefotetan with 3.8% of tautomer. The solution obtained is fed over 41 min through a 32 mm diameter column containing 240 ml of suitably conditioned HP 20 SS resin. Elution is carried out with osmotized water. 250 g of a rich fraction containing ≦0.2% tautomer are collected in about 60 min.

The solution obtained is acidified at 20° C. to pH 3.4-3.5 with 10% phosphoric acid and then maintained under vacuum at 20° C. for 30 min. The temperature is raised to 30-35° C. until the foam disappears, then 5% HCl is added to pH 2.5. The solution is maintained under agitation at 30° C. for one hour. On termination the pH is further lowered to 1.5 with 5% HCl. After 30 min at 30° C. the solution is cooled to 5° C. and agitated at that temperature for 2 hours.

The mixture is filtered, the product washed with 50 ml 5% HCl and then with 100 ml of osmotized water, both precooled to 5° C. The product is dried at 45°-50° C. until K.F≦2.5%.

Yield: 12.33 g with titre (HPLC) as such of 97.4%, a tautomer (II) content (HPLC) less than 0.5% and with no residual solvents.

What is claimed is:

1. A process for obtaining cefotetan of formula 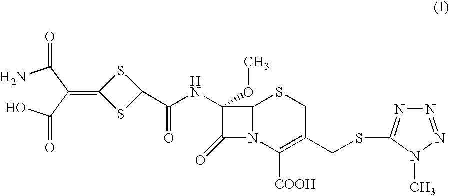
containing up to 0.5% of the tautomer of formula 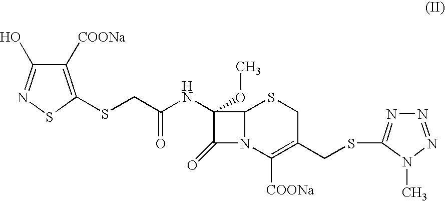
in its acid form with K.F. up to 2.5%, dry content at least 99.0% and totally free of solvents, characterised by slowly and gradually acidifying an aqueous solution of cefotetan at pH 7.5 containing carbon dioxide and up to 5% of the said tautomer of formula (II), with an acid chosen from the group consisting of dilute hydrochloric acid and dilute phosphoric acid to pH 3.4, while maintaining the solution under agitation and under vacuum at a temperature between 20° and 35° C. in order to remove the carbon dioxide, said solution then being further acidified to pH 1.5 to allow crystallization of the cefotetan of formula (I), which is filtered off and washed, this cefotetan then being returned to solution at pH less than 7, the solution being adsorbed on HP 20 SS resin and then eluted with osmotized water to obtain a solution from which the aforesaid cefotetan of formula (I) is precipitated by acidification to pH 1.5 with dilute hydrochloric acid.

2. Cefotetan of formula (I) 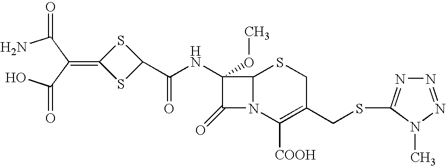
which is totally solvent-free.
